(19) United States
(12) Patent Application Publication
(10) Pub. No.: US 2002/0022778 A1
(43) Pub. Date: Feb. 21, 2002

(54) Imaging technique and magnetic resonance tomograph

(76) Inventors: Stefan Wiese, Koln (DE); Nadim Joni Shah, Julich (DE)

Correspondence Address: Ashley I. Pezzner, Esquire, CONNOLLY BOVE LODGE & HUTZ LLP, 1220 Market Street, P.O. Box 2207, Wilmington, DE, 19899, US

(21) Appl. No.: 09/742,842

(22) Filed: Jun. 27, 2001

(30) Foreign Application Priority Data
Dec. 24, 1999 (DE) 199 62 850.5

(51) Int. Cl. A61B 5/055

(52) U.S. Cl. 600/410; 324/307; 324/309

(57) ABSTRACT

The invention relates to a method to operate a nuclear resonance tomograph involving the suppression of image artifacts that are brought about by interfering nuclear spins, a transverse magnetization being generated in a volume of interest by applying a slice-selective HF excitation pulse to nuclear spins in the presence of a first magnetic field gradient and a polarizing magnetic field, and a spin echo being subsequently generated by means of at least a second slice-selective HF pulse in conjunction with a magnetic field gradient.

The method is carried out according to the invention in such a way that, outside of at least one target volume to be examined, the transversal magnetization after the application of the HF excitation pulse is disturbed by at least one additional magnetic field gradient.

### DESCRIPTION

The invention relates to an imaging technique in which high-frequency pulses are emitted and at least one magnetic gradient field is applied in order to select at least one slice or volume of interest in which a nuclear magnetic resonance is excited and determined as a measuring signal.

The invention also relates to a magnetic resonance tomograph having a means to emit high-frequency pulses, a means to apply at least one magnetic gradient field and a means to detect a measuring signal.

Nuclear magnetic resonance tomography (NMR) (also known as zeugmatography) is employed, among other things, to obtain spectroscopic information about a substance. A combination of nuclear magnetic resonance tomography with the techniques of magnetic resonance imaging (MRI) provides a spatial image of the chemical composition of the substance.

In nuclear resonance tomography, atom nuclei having a magnetic momentum are oriented by a magnetic field applied from the outside. In this process, the nuclei execute a precession having a characteristic angular frequency (Larmor frequency) around the direction of the magnetic field. The Larmor frequency depends on the strength of the magnetic field and on the magnetic properties of the substance, particularly on the magnetic constant γ of the nucleus. The gyromagnetic constant γ is a characteristic quantity for every type of atom. The atom nuclei have a magnetic momentum μ=γ×p wherein p stands for the angular momentum (spin) of the nucleus.

In nuclear resonance tomography, a substance or a person to be examined is subjected to a uniform magnetic field. This uniform magnetic field is also called a polarization field Bo and the axis of the uniform magnetic field is called the z axis. With their characteristic Larmor frequency, the individual magnetic momentums of the spin in the tissue precede around the axis of the uniform magnetic field.

This generates a net magnetic momentum M in the direction of the polarization field whereby, however, the randomly oriented magnetic components cancel each other out in the plane perpendicular to this (the x-y plane). After the uniform magnetic field has been applied, an excitation field B₁ is additionally generated. This excitation field B₁ is polarized in the x-y plane and it has a frequency that is close to the Larmor frequency. As a result, the net magnetic momentum M can be tilted into the x-y plane, so that a transverse magnetic momentum M_{xy} is created. The transverse magnetic momentum M_{xy} rotates in the x-y plane.

By varying the time of the excitation field, several temporal sequences of the transverse magnetic momentum M_{xy} can be generated. For this purpose, a gradient field is applied through which a slice is selected.

Particularly in medical research, there is a need for information about anatomical structures, spatial distributions of substances as well as about brain activity or, in a broader sense, for information about blood flow or changes in the concentration of deoxyhemoglobin in the organs of animals and humans.

Magnetic resonance spectroscopy (MRS) makes it possible to measure the spatial density distribution of certain chemical components in a material, especially in biological tissue.

Rapid magnetic resonance imaging (MRI), in conjunction with magnetic resonance spectroscopy (MRS), allows an examination of local distributions of metabolic processes. For instance, as a function of brain activity, regional hemodynamics involving changes in the blood volumes and blood states as well as changes in the metabolism can be determined in vivo.

NMR imaging methods select slices or volumes that yield a measuring signal under appropriate irradiation with high-frequency pulses and under the application of magnetic gradient fields; this measuring signal is digitized and stored in a one-dimensional or multi-dimensional field in a measuring computer.

The desired image information is then acquired (reconstructed) from the raw data collected; this is done by means of a temporal or spatial Fourier transform.

A reconstructed slice image consists of pixels (picture elements), and a volume data set consists of voxels (volume picture elements). A pixel is a two-dimensional picture element, for instance, a rectangle. The image is made up of pixels. For metrological reasons, voxels and pixels do not exhibit any sharp boundaries. The dimensions of a pixel are normally in the order of magnitude of 1 mm², and those of a voxel are in the order of magnitude of 1 mm². The geometries and extents can vary.

Seeing that, for experimental reasons, it is never possible to assume a strictly two-dimensional plane in the case of tomographs, the term voxel (volume picture element) is often employed here as well, and it indicates that the image planes have a certain thickness.

Due to the large differences in the signal intensities of individual chemical substances, due to chemical shifting as well as to movements of the object being measured, localization artifacts can occur during imaging and spectroscopy.

Particularly in brain examinations, it is necessary to suppress signals from substances that are located outside of the brain. In the case of magnetic resonance with protons (¹H), these are substances, for example, subcutaneous lipids, that contain ¹H. Lipids are a component of cell membranes and consequently are present in all biological substances. In view of their high concentration, subcutaneous lipids have a strong signal, which masks signals from metabolites.

Lipids cover a rather broad frequency range that coincides with that of most metabolites. In spectroscopic examinations of the brain, the suppression of signals from substances that are located outside of the brain—which is also referred to as lipid suppression—is necessary because the signals thus generated can be much stronger than the signals in the brain regions to be examined.

In view of the fact that the lipids in the human head are primarily to be found in the periphery of the skull, one possibility of lipid suppression is to not even excite the nuclear spins in the periphery. A spatially localized spectrum is obtained by means of signal suppression in regions located outside of a volume to be examined.

A known technique is the STEAM method, with which a large volume can be excited that is divided into voxels by means of gradients. The STEAM method is described in the following articles:

Garnot J. (1986): Selected volume excitation using stimulated echoes (VEST). Applications to spatially localized spectroscopy and imaging; J. Magn. Reson. 70: pages 488 to 492;

Kimmich R., Hoepfel D. (1987): Volume selective multipulse spin echo spectroscopy. J. Magn. Res. 72: pages 379 to 384;

Frahm J., Merboldt K. D., Haenicke W. (1987): Localized proton spectroscopy using stimulated echoes. J. Magn. Res. 72: pages 502 to 508.

The STEAM method entails the disadvantage that a signal-to-noise ratio (SNR) diminishes to half of its value.

Another volume localization method called PRESS is disclosed in U.S. Pat. No. 4,480,228 by Bottomley P. A. (1984) titled “Selective volume method for performing localized NMR spectroscopy”.

Another known volume localization method is presented by Ordidge R. J., Bendall M. R., Gordon R. E., Connelly A. in an article titled “Volume selection for in-vivo biological spectroscopy” in the book titled Magnetic Resonance in Biology and Medicine, published by Govil, Khetrapal and Saran, New Delhi, India, Tate McGraw-Hill Publishing Co. Ltd., page 387 (1985).

The known volume localization methods all have the disadvantage that an examination of the spatial distribution of chemical substances is only possibly to a limited extent. Another disadvantage of the known methods is a limitation of the signal suppression outside of a target volume due to imperfections in the slice selection, whereby a slight lipid suppression is achieved and/or whereby it is only possible to select rectangular target volumes.

Particularly in the case of short echo times, it is difficult to avoid interferences from signals of subcutaneous lipids that have a short relaxation time T₂*.

It is a known procedure to select long echo times is order to reduce the detrimental effects of lipid impurities.

Examples of embodiments can be found in the following articles:

Frahm J., Bruhn H., Gyngell M. L., Merboldt K. D., Haenicke W., Sauter R. (1989): Localized high-resolution proton NMR spectroscopy using stimulated echoes. Initial application to human brain in vivo. Magn. Reson. Med.: pages 79 to 93;

Frahm J., Bruhn H., Haenicke W., Merboldt K. D., Mursch K., Markakais E. (1991): Localized proton NMR spectroscopy of brain tumors using short-echo time TEAM sequences. J. Comp. Assist. Tomogr.: 15 (6), pages 915 to 922,

Moonen C. T. W., Sobering G., van Zijl P. C. M., Gillen J., von Kienlin M., Bizzi A. (1992): Proton spectroscopic imaging of human brain. J. Magn. Reson., 98 (3): pages 556 to 575.

Spectroscopic imaging entails the problem that the volumes of interest (VOI) extend close to regions containing high concentrations of peripheral, subcutaneous lipids.

Moreover, a systematic spatial localization of the target volume to be examined by a combination of volume excitations and spatial suppression of the surrounding regions is known from the following publications:

Connelly A., Counsell C., Lohmann J. A. B., Ordidge R. (1988): Outer volume suppressed image related in vivo spectroscopy (Osiris): A high sensitivity localization technique. J. Magn. Reson. 78 (3): pages 519 to 525;

Singh S., Rutt B. K., Henkelmann R. M. (1990). Projection presaturation: A fast and accurate technique for multidimensional spatial localization. J. Magn. Reson. 87: pages 567 to 583;

Duyn J. H., Gillen J., Sobering G., van Zijl P. C. M., Moonen C. T. W. (1993): Multisection proton MR spectroscopic imaging of the brain. Radiology 188: pages 277 to 282;

Shungu D., Glickson J. D. (1994): Band-Selective Spin Echoes for in vivo Localized ¹H NMR Spectroscopy. J. Magn. Reson. Med., 32 (3): pages 277 to 284,

Chen Y. J., Rachamadugu S., Fernandez E. J. (1997): Three-dimensional outer volume suppression for short echo time in vivo ¹H spectroscopic imaging in rat brain. Magn. Reson. Imag. 15: pages 839 to 845;

Posse S., Schuhknecht B., Smith B., van Zijl P. C. M., Herschkowitz N., Moonen C. T. W. (1993): “Short-echo-time proton spectroscopic imaging”, J. of Comp. Assist. Tomogr., 15: pages 839 to 845.

The article by Adalsteinsson E., Irarrazabal P., Spielman D. M., Macovski A. (1995) titled “Three-Dimensional Spectroscopic Imaging with Time-Varying Gradients”; Magn. Reson. Med., 33: pages 461 to 466, describes three-dimensional spectroscopic imaging with lipid suppression by global inversion of the signal utilizing differences in the longitudinal relaxation between individual chemical substances.

An improved water and lipid suppression by means of spectral-selective dephasing pulses is known as the BASING method. A description of the BASING method can be found in the article by Star-Lack J., Nelson S. J., Kurhanewicz J., Huang R., Vigneron D. (1997) titled “Improved water and lipid suppression for 3D PRESS CSI using RF Band-Selective Inversion with Gradient Dephasing (BASING)”. Magn. Reson. Med. 38: pages 311 to 321.

The BASING method comprises a frequency-selective refocusing pulse in conjunction with immediately preceding and following gradient pulses having opposite signs, which leads to dephasing.

It is also a known procedure to employ functional nuclear magnetic resonance to examine neuronal activation. Neuronal activation is manifested by an increase of the blood flow into activated regions of the brain, whereby there is a drop in the concentration of deoxyhemoglobin in the blood. Deoxyhemoglobin (DOH) is a paramagnetic substance that reduces the magnetic field homogeneity and thus accelerates signal relaxation. If the DOH concentration decreases owing to brain activity, then signal relaxation is slowed down in the active regions of the brain. It is primarily the protons of hydrogen in water that are excited. It is possible to localize the brain activity by conducting an examination with functional NMR methods that measure the NMR signal with a time delay (echo time). This is also referred to as a susceptibility-sensitive measurement. The biological mechanism of action is known in the literature under the name BOLD effect (Blood Oxygen Level Dependent effect) and, in susceptibility-sensitive magnetic resonance measurements at a field strength of a static magnetic field of, for example, 1.5 tesla, it leads to a fluctuation of up to about 5% in the image brightness in activated regions of the brain. Instead of the endogenous contrast agent DOH, other contrast agents that cause a change in the susceptibility can also be used. Here, too, lipid signals are suppressed. Preference is given to using a frequency-selective lipid presaturation.

Echo planar spectroscopic imaging (EPSI) is elaborated upon in the article by P. Mansfield: Magn. Reson. Med. 1, page 370, 1984.

In the brain and in other organs, residual signals from peripheral regions having a high concentration of lipids can cause considerable spectral artifacts that limit the interpretation and quantification procedures.

A method entailing improved lipid suppression using a stimulated echo is presented in U.S. Pat. No. 5,709,208 by Posse S., LeBihan D. titled “Method and System for Multidimensional Localization and for Rapid Magnetic Resonance Spectroscopic Imaging”. This method allows a more flexible selection of the suppressed volume as well as faster data acquisition in order to generate spectroscopic images. However, it entails the drawback that the useful signal drops by 50%. The volume to be examined is selected by means of a spatial presuppression frequency, followed by a stimulated echo sequence and a suppression sequence repeated during the mixing time (TM). The presuppression sequence includes a spatial suppression sequence to selectively saturate selected slices that intersect a slice that is selected by the stimulated echo sequence. Such repeated spatial dephasing, however, is not possible with the spin echo method.

The invention is based on the objective of improving the known methods so that they can be performed more rapidly and so that interfering signals can be effectively suppressed.

This objective is achieved according to the invention in that a time sequence of the measuring signal is detected as the relaxation signal and in that at least one image signal is encoded during the detection of the relaxation signal.

The invention also has the objective of creating a magnetic resonance tomograph suitable for carrying out this method.

According to the invention, this objective is achieved in that a magnetic resonance tomograph of this type is configured in such a way that it has a means to detect a time sequence of the measuring signal as the relaxation signal as well as a means to encode at least one image signal whereby the encoding means is configured in such a way that it encodes the image signal during the detection of the relaxation signal.

A particularly fast and reliable embodiment of the method is characterized in that the gradient fields are selected during a spatial encoding in such a manner that at least one time derivative of at least one k vector in a k space is essentially constant.

An advantageous embodiment of this execution method is characterized in that, during a spatial encoding, a gradient field is kept essentially constant.

In order to further enhance the reliability of the data acquisition, it is advantageous to keep all gradient fields essentially constant during the spatial encoding. This variant of the method has the advantage that it can be carried out without much experimental effort.

Another, likewise advantageous embodiment of the invention is characterized in that at least one gradient field is varied over time and in that at least one detection interval is varied over time. This variant of the method has the advantage that the signal-to-noise ratio is particularly high.

This variant of the invention makes it possible to achieve detection constancy by means of a suitable adaptation of the interval lengths.

Additional advantages, special features and practical refinements of the invention ensue from the following presentation of the example of a preferred embodiment making reference to the drawings.

The drawings show the following:

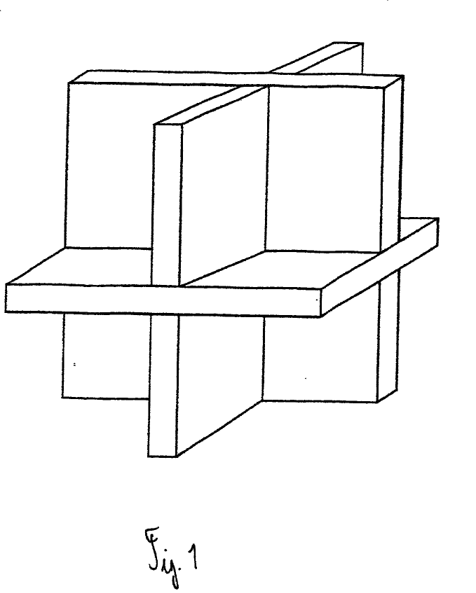

FIG. 1 a depiction of an area selected for signal excitations;

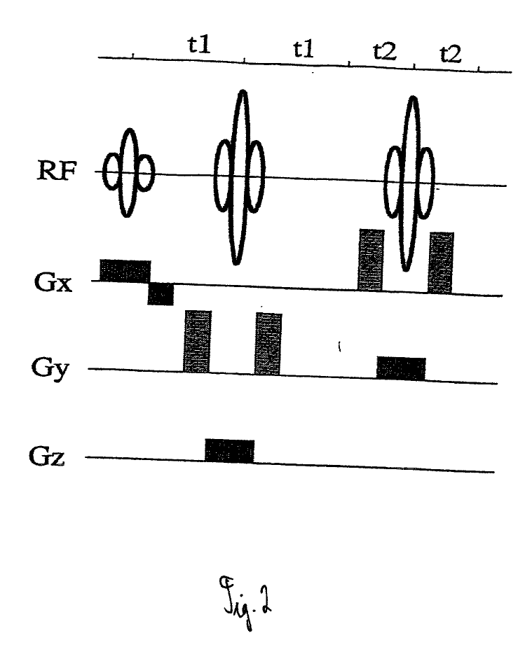

FIG. 2 an excitation sequence suitable for carrying out the method according to the invention; and

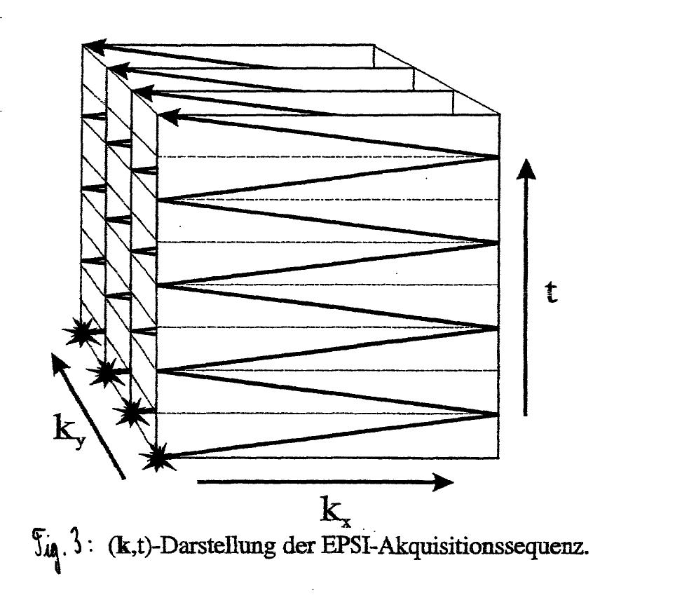

FIG. 3 a (k, t) depiction of an acquisition sequence suitable for the acquisition according to the invention of measured data.

FIGS. 1 to 3 show preferred embodiments of a method according to the invention for rapid spectroscopic metabolite imaging using a nuclear spin tomograph, comprising a volume-selective signal excitation (PRESS=Point RESolved Spectroscopy) with subsequent spatial-spectral encoding (EPSI=echo planar spectroscopic imaging).

In magnetic resonance spectroscopy (MRS), sectional images are generated with a predefined grid of N_{y} lines and Nₓ columns (CSI=chemical shift imaging). The preferred process steps are presented below:

1) First of all, resonant nuclear spins located in a volume of interest of the sample and polarized in the presence of an external magnetic field B₀=B₀e_{z} are excited by means of suitable RF irradiation (RF=radio frequency) in order to generate a signal. The magnetization M, which is altogether influenced by the nuclear spin, hereby acquires a measurable component M_{xy} that is orthogonal to B₀ and that precedes with the angular velocity ω=−γB₀.

2) Subsequently, the signal is spatially encoded through the brief application of magnetic field gradients G=ΔB₀/Δr, whose purpose is to vary the external magnetic field linearly to the site r. As a result, the resonant nuclear spins precede for a brief time with an additional angular frequency Δω(r)=−γGr and emit a phase-modulated MR signal after the gradient G has been switched off.

3) This modulated MR signal is then scanned for a sufficiently long time, that is to say, about as long as necessary for M_{xy} to become completely dephased, and at sufficiently frequent time intervals.

4) Steps two and three are repeated as many times as the sectional image is supposed to have grid points, in other words, (N_{y}·Nₓ) times. With each repetition, the gradient strength G or the time duration of the application is varied, as is needed for a correct spatial encoding.

5) A digital computer is then employed to further process the data points thus acquired and ultimately the sectional images are computed.

The execution, however, can also be completed with just some of the steps described. For instance, the second and fourth steps can be dispensed with if spatially resolved encoding is not needed. This results in spatially resolved frequency spectra on the basis of which the relative concentration of individual chemical components can be computed. These can be distinguished because the effective magnetic field at the site of a nucleus and thus also its precession frequency are a function of its parent molecule, which shields the external magnetic field to a greater or lesser extent.

When it comes to the examination of biological tissue, it is most advantageous to select protons as the resonant nuclei. In this context, the very strong signals of the water and of the lipids at concentrations in the double-digit molar range are to be suppressed so that the metabolic products (metabolites) of interest can be detected in the millimolar range. The signal of water protons is relatively easy to suppress since it is present virtually isolated in the frequency spectrum, as a result of which it can be eliminated by appropriate RF irradiation. There are combinations of CHESS pulses (CHESS=CHEmical Shift Selective) with which suppression factors of up to 3000 can be attained,

In order to reduce the measuring duration by more than one order of magnitude in spatially resolved spectroscopy, the phase encoding can be partially combined with the readout of the MR signal. Echo-Planar Spectroscopic Imaging (EPSI) is considered to be difficult to implement on clinical nuclear spin tomographs, in addition to which it makes high requirements of the quality of the hardware components, particularly of the homogeneity of the main magnetic field B₀. The EPSI method has not met with widespread acceptance, but this could change as a result of the present invention. The advantage lies in a measuring duration that is shortened by the factor Nₓ.

An advantageous execution of the echo-planar spectroscopic imaging method involves, in particular, a repeated echo-planar spectroscopic imaging method consisting of the repeated application of two-dimensional echo-planar image encoding. Spatial encoding takes place within the shortest possible time span that is repeated multiple times during a signal drop and preferably amounts to 20 ms to 100 ms. The multiple repetition of the echo-planar encoding during a signal drop serves to depict a course of the signal drop in the sequence of reconstructed individual images.

A PRESS excitation serves to specifically excite a sample volume that is defined as a sectional parallelepiped of three orthogonal slices. The nuclear spins within this target volume generate the MR signal from a double-spin echo, corresponding to the three slice-selective RF pulses, on the basis of which PRESS is built up:

90°−t₁₋180°−t₁−spin echo−t₂₋180°−t₂−measurement.

Spins that lie outside of the target volume but that have been subjected to the 90° pulse at best experience another 180° pulse and are otherwise dephased by the requisite sliceselection gradients. Spins that have not been subjected to the 90° pulse do not lead to any measurable signal, even if they have experienced one or both 180° pulses.

Unsharpness of the slice profiles of the 180° pulses—as a result of which undesired MR signals from outside of the volume of interest can occur—should be avoided. To this end, one possibility is a dephasing of the signal (crushing). Crushing can be achieved most easily in that the slice-selection gradients of the two 180° pulses last longer than would otherwise be necessary. The slice-selection gradients, however, still have to be arranged symmetrically around the 180° pulse in order not to destroy the spin re-phasing.

Another improvement can be achieved by carrying out the crushing with markedly stronger gradients that are orthogonal to the slice-selection gradients. This precludes the possibility of re-phasing of undesired stimulated echoes.

Subsequently, a signal excitation, especially a PRESS signal excitation, is read out by means of spatial-spectral encoding (EPSI). Here, in a (k, t) diagram, an entire (kₓ, t) slice is acquired for each PRESS excitation. Which slice this refers to is selected immediately after the PRESS excitation by means of a phase-encoding gradient in the k_{y} direction.

Therefore, in order to measure a (kₓ, t) slice, the signal only has to be excited once, in contrast to the commonly employed spectroscopic imaging, where Nₓ signal excitations would have been necessary for this purpose. Once this EPSI read-out is complete, the measured data is re-interpreted in a suitable manner, namely, as (kₓ, k_{y}) slices at various points in time t. Formally, this is done by rearranging the measured data. Afterwards, the data can be further processed using familiar methods of conventional spectroscopic imaging.

The coordinates (kₓ, k_{y}) shown are only given as examples. The person skilled in the art can select suitable (kₓ, k_{y}) for every examination.

1. An imaging method in which high-frequency pulses are emitted and at least one magnetic gradient field is applied in order to select at least one slice or volume of interest in which a nuclear magnetic resonance is excited and a measuring signal is ascertained, characterized in that a time sequence of the measuring signal is detected as the relaxation signal and in that at least one image signal is encoded during the detection of the relaxation signal.

2. The method of claim 1, characterized in that the gradient fields are selected during a spatial encoding in such a manner that at least one time derivative of at least one k vector in a k space is essentially constant.

3. The method of one or both of claims 1 and 2, characterized in that, during a spatial encoding, one gradient field is kept essentially constant.

4. The method of claim 3, characterized in that, during a spatial encoding, all of the gradient fields are kept essentially constant.

5. The method of one or both of claims 1 and 2, characterized in that at least one gradient field is varied over the course of time and in that detection intervals occurring at different times have lengths that differ from each other.

6. The method of claim 5, characterized in that the detection intervals are selected in such a way that they fulfill the condition kₘₐₓΔx<π.

7. The method of one or more of claims 1 through 6, characterized in that a dephasing of the signal takes place outside of a volume of interest.

8. The method of claim 7, characterized in that the dephasing is carried out with essentially orthogonal gradients.

9. The method of one or more of claims 1 through 8, characterized in that a multiple spin-echo signal is generated in a volume of interest.

10. The method of claim 9, characterized in that a double spin echo is generated in a volume of interest.

11. The method of one or more of claims 1 through 10, characterized in that at least one signal excitation is read out by means of spatial-spectral encoding.

12. The method of claim 11, characterized in that the spatial-spectral encoding is performed in such a way that a defined (kₓ, t) area is acquired in a (k, t) diagram for an excitation.

13. The method of claim 12, characterized in that, in the (k, t) diagram, a (kₓ, t) slice is acquired for the excitation.

14. The method of claim 13, characterized in that the (kₓ, t) slice is excited by a phaseencoding gradient in the k_{y} direction.

15. The method of claim 14, characterized in that the (kₓ, t) slice is selected immediately after the excitation by the phase-encoding gradient in the k_{y} direction.

16. The method of one or more of claims 1 through 15, characterized in that a PRESS sequence with a 90° pulse, an 180° pulse and another 180° pulse is employed.

17. A magnetic resonance tomograph with a means to emit high-frequency pulses, a means to apply at least one magnetic gradient field and a means to detect a measuring signal, characterized in that the magnetic resonance tomograph has a means to detect a measuring signal as the relaxation signal and a means to encode at least one image signal, whereby the encoding means is designed in such a way that it encodes the image signal during the detection of the relaxation signal.
